(19) United States
(12) Patent Application Publication
(10) Pub. No.: US 2005/0186274 A1
(43) Pub. Date: Aug. 25, 2005

(54) Multilayer tablet

(75) Inventor: Anja Kohlrausch, Biberach (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/056,081

(22) Filed: Feb. 10, 2005

(30) Foreign Application Priority Data
Feb. 20, 2004 (DE) 10 2004 008 804

(51) Int. Cl. A61K 9/20

(52) U.S. Cl. 424/464

(57) ABSTRACT

A multilayer tablet comprises a first layer formulated for instant release of the angiotensin II receptor antagonist telmisartan from a dissolving tablet matrix, a second layer formulated for instant release of the angiotensin converting enzyme inhibitor ramipril and optionally a diuretic from a disintegrating tablet matrix, and, optionally, a third layer formulated for instant release of a diuretic like hydrochlorothiazide from a fast disintegrating tablet matrix.

### RELATED APPLICATIONS

This application claims priority of German Application No. 10 2004 008 804, filed Feb. 20, 2004.

### FIELD OF THE INVENTION

The present invention relates to a pharmaceutical tablet comprising a first layer of the angiotensin II receptor antagonist telmisartan in a dissolving tablet matrix, a second layer of the angiotensin converting enzyme (ACE) inhibitor ramipril alone or together with a diuretic in a disintegrating tablet matrix, and optionally a third layer of a diuretic like hydrochlorothiazide in a fast disintegrating tablet matrix.

### BACKGROUND OF THE INVENTION

Telmisartan is an angiotensin II receptor antagonist developed for the treatment of hypertension and other medical indications as disclosed in EP-A-502314. Its chemical name is 4′-[2-n-propyl-4-methyl-6-(1-methylbenzimidazol-2-yl)-benzimidazol-1-ylmethyl]-biphenyl-2-carboxylic acid having the following structure:

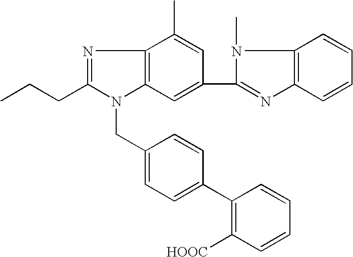

Telmisartan is manufactured and supplied in the free acid form. It is characterized by its very poor solubility in aqueous systems at the physiological pH range of the gastrointestinal tract of between pH 1 to 7. As disclosed in WO 00/43370, crystalline telmisartan exists in two polymorphic forms having different melting points. Under the influence of heat and humidity, the lower melting polymorph B transforms irreversibly into the higher melting polymorph A.

Ramipril disclosed in EP-A-079022 is a long-acting ACE inhibitor with the chemical name (2S,3aS,6aS)-1[(S)-N-[(S)-1-Carboxy-3-phenylpropyl]alanyl]-octahydro-cyclopenta[b]pyrrole-2-carboxylic acid, 1-ethyl ester having the following structure:

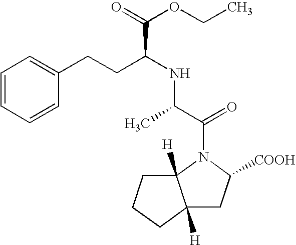

It inhibits the conversion of angiotensin I into angiotensin II as well as the breakdown of the active vasodilator bradykinin. Both of these activities lead to vasodilation. It is being used in the treatment of hypertension and congestive heart failure and its active metabolite is the free acid ramiprilat, which is obtained in vivo upon administration of ramipril. Additionally ramipril has been suggested to effect a pronounced inhibition of ACE in tissues resulting in organ protective effects such as the heart, lung, and kidney

Diuretics are therapeutic agents used in the treatment of edema and hypertension. Occasionally they are combined with anti-hypertensive agents acting on the basis of a different mode of action to achieve synergistic therapeutic efficacy in the treatment of hypertension. A preferred diuretic is hydrochlorothiazide (HCTZ). The chemical name of HCTZ is 6-chloro-3,4-dihydro-2H-1,2,4-benzothiadiazine-7-sulfonamide-1,1-dioxide having the following structure:

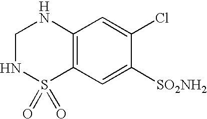

### OBJECT OF THE INVENTION

The mechanisms of action of telmisartan and ramipril are considered to cooperate favourably in the treatment or prevention of conditions such as stroke, myocardial infarction, transient ischaemic attack, cardiovascular disease, diabetes, cognitive decline and dementia. As this assumption gains supported from an increasing amount of clinical data, there is an increasing desire for a fixed dose combination drug comprising the active ingredients telmisartan, ramipril and, optionally, a diuretic such as hydrochlorothiazide. However, both telmisartan and ramipril are chemical compounds which are difficult to handle. Therefore, an oral fixed dose combination drug which combines the features of pharmacologic efficacy, adequate drug stability and a reliable and robust method of manufacture has to overcome a number of technical problems. It is an object of the present invention to provide such a fixed dose combination drug.

There are various types of fixed dose dosage forms conceivable but it cannot be predicted which of these dosage forms best combines product stability, pharmacological efficacy and reliable manufacture. Examples of such dosage forms are oral osmotic systems (OROS), coated tablets, matrix tablets, press-coated tablets, multilayer tablets and the like. The present invention is based on the recognition, that the dosage form, which combines adequate drug stability, optimum drug release of both active ingredients, pharmacological efficacy and reliable manufacture for a combination of telmisartan and ramipril best is a multilayer tablet.

Generally, a fixed-dose combination of drugs intended for instant release is prepared by either making a powder mixture or a co-granulate of the two active ingredients with the necessary excipients, normally keeping the basic formulation of the corresponding mono-drug preparation and simply adding the second drug component.

With a combination of telmisartan and ramipril, this approach does not appear feasible due to the incompatibility of ramipril with components of the conventional telmisartan formulations. When including the diuretic HCTZ or the ACE inhibitor ramipril in a combination, a reduced dissolution rate of HCTZ from a dissolving matrix as compared with dissolution from a disintegrating tablet is observed. Coating HCTZ or ramipril particles in a fluidized-bed granulator with a polymer solution containing water soluble polymers like hydroxypropylcellulose, hydroxypropylmethylcellulose or polyvinylpyrrolidone to reduce the contact surface area of HCTZ particles with the telmisartan or ramipril formulation during mixing and compressing does not reduce the contact area of HCTZ with the telmisartan or ramipril formulation in a compressed tablet to a degree sufficient to achieve the desired prolonged shelf life. Furthermore, the dissolution rate of HCTZ from tablets comprising coated HCTZ appears further reduced due to the gel-forming properties of the polymer.

Another approach is to produce separate film-coated tablets for telmisartan, ramipril and, optionally, HCTZ in such a size and shape that these can be filled into a capsule. By dividing the doses into small tablets for telmisartan, ramipril and, optionally, HCTZ, the drug dissolution rate of telmisartan and ramipril is found to be reduced compared to the single entities due to a lag-time effect of the large capsule shells. Furthermore, with regard to patients' compliance, a zero long capsule is not deemed reliable.

### SUMMARY OF THE INVENTION

In accordance with the present invention problems associated with the preparation of a fixed dose combination drug comprising telmisartan, ramipril and, optionally, a diuretic can best be handled by means of a multilayer pharmaceutical tablet comprising a first layer of telmisartan, preferably in substantially amorphous form, in a dissolving tablet matrix and a second layer of ramipril alone or ramipril together with a diuretic such as HCTZ in a disintegrating tablet matrix. Alternatively, the tablet may contain a third layer comprising the diuretic in a disintegrating tablet matrix.

The tablet according to the present invention provides a largely pH-independent dissolution of the poorly water-soluble telmisartan, thereby facilitating dissolution of the drug at a physiological pH level, and adequate stability and drug release of ramipril. In combination with a diuretic it provides for instant release of the diuretic from a fast disintegrating matrix. The tablet structure also overcomes the stability problem caused by the incompatibility of diuretics like HCTZ with basic constituents of telmisartan formulations and the stability problem caused by the incompatibility of ramipril with basic constituents of telmisartan.

### DEFINITIONS

As used herein, the term “substantially amorphous” refers to a product comprising amorphous constituents in a proportion of at least 90%, preferably at least 95%, as determined by X-ray powder diffraction measurement.

The term “dissolving tablet matrix” refers to a pharmaceutical tablet base formulation having instant release (fast dissolution) characteristics that readily dissolves in a physiological aqueous medium.

The term “diuretic” refers to thiazide and thiazide-analogue diuretics like hydrochlorothiazide (HCTZ), clopamide, xipamide or chlorotalidone, and any other diuretic suitable in the treatment of hypertension like furosemide and piretanide, and combinations thereof with amiloride and triamteren.

The term “disintegrating tablet matrix” refers to a pharmaceutical tablet base formulation having instant release characteristics that readily disintegrates in a physiological aqueous medium.

### DETAILED DESCRIPTION OF THE INVENTION

A fixed dose combination according to the present invention represents a pharmaceutical multilayer tablet comprising a first layer of telmisartan in substantially amorphous form, a second layer of ramipril alone or ramipril together with a diuretic in a disintegrating tablet matrix, or optionally a third layer of a diuretic in a disintegrating tablet matrix.

The active ingredient telmisartan is generally supplied in its free acid form, although pharmaceutically acceptable salts such as the sodium salt may also be used. Since during subsequent processing telmisartan is normally dissolved and transformed into a substantially amorphous form, its initial crystal morphology and particle size are of little importance for the physical and biopharmaceutical properties of the multilayer tablet formulation obtained. It is, however, preferred to remove agglomerates from the starting material, e.g. by sieving, in order to facilitate wetting and dissolution during further processing.

Substantially amorphous telmisartan may be produced by any suitable method known to those skilled in the art, for instance, by freeze drying of aqueous solutions, coating of carrier particles in a fluidized bed, and solvent deposition on sugar pellets or other carriers. Preferably, however, the substantially amorphous telmisartan is prepared by the specific spray-drying method described in WO03/059327.

Ramipril is supplied as a free ester or stabilized with a polymeric coating as described in EP-A-317878. Examples of polymers suitable for the protective coating are cellulose derivatives such as hydroxyproplycellulose, hydroxypropylmethyl-cellulose, hydroxypropylmethylcellulose phthalate, hydroxyethylcellulose, ethylcellulose, cellulose acetate phthalate, cellulose acetate, polyvinyl acetate phthalate, polyvinylpyrrolidone, cationic and anionic polymers, copolymer with neutral character based on poly(meth)acrylic esters (Eudragit®(R) E, Eudragit®(R) E 30 D), anionic polymer of methacrylic acid and methyl methacrylate (Eudragit®(R) L or S, Eudragit®(R) L 30 D) and gelatin.

The diuretic is usually employed as a fine-crystalline powder, optionally in fine-milled, peg-milled or micronized form. For instance, the particle size distribution of hydrochlorothiazide, as determined by the method of laser light scattering in a dry dispersion system (Sympatec Helos/Rodos, focal length 100 mm) is preferably as follows:
d₁₀: ≦20 μm, preferably 2 to 10 μm
d₅₀: 5 to 50 μm, preferably 10 to 30 μm
d₉₀: 20 to 100 μm, preferably 40 to 80 μm

A multilayer tablet according to the present invention generally contains 10 to 160 mg, preferably 20 to 80 mg or 40 to 80 mg, of telmisartan; 1 to 20 mg, preferably 5 to 10 mg, of ramipril; and 6.25 to 50 mg, preferably 12.5 to 25 mg, of a diuretic such as HCTZ.

Presently preferred forms are multilayer tablets comprising 20/10 mg, 40/10 mg, 80/10 mg, 20/5 mg, 40/5 mg, 80/5 mg, 20/2.5 mg, 40/2.5 mg and 80/2.5 mg of telmisartan and ramipril, respectively. The preferred amounts of diuretic are 12.5 mg or 25 mg.

The first tablet layer contains telmisartan in substantially amorphous form dispersed in a dissolving tablet matrix having instant release (fast dissolution) characteristics. The dissolving tablet matrix may have neutral or basic properties, although a basic tablet matrix is preferred.

In such a preferred embodiment, the dissolving matrix of the telmisartan layer comprises a basic agent, a water-soluble diluent and, optionally, other excipients and adjuvants.

Specific examples of suitable basic agents are alkali metal hydroxides such as NaOH and KOH; basic amino acids such as arginine and lysine; and meglumine (N-methyl-D-glucamine), NaOH and meglumine being preferred.

Specific examples of suitable water-soluble diluents are carbohydrates such as monosaccharides like glucose; oligosaccharides like sucrose, anhydrous lactose and lactose monohydrate; and sugar alcohols like sorbitol, mannitol, erythrol and xylitol. Sorbitol is a preferred diluent.

The other excipients and/or adjuvants are, for instance, selected from binders, carriers, fillers, lubricants, flow control agents, crystallization retarders, solubilizers, coloring agents, pH control agents, surfactants and emulsifiers, specific examples of which are given below in connection with the second tablet layer composition. The excipients and/or adjuvants for the first tablet layer composition are preferably chosen such that a non-acidic, fast dissolving tablet matrix is obtained.

The first tablet layer composition generally comprises 3 to 50 wt. %, preferably 5 to 35 wt. %, of active ingredient; 0.25 to 20 wt. %, preferably 0.40 to 15 wt. %, of basic agent; and 30 to 95 wt. %, preferably 60 to 80 wt. % of water-soluble diluent (filler).

Other (optional) constituents may, for instance, be chosen from one or more of the following excipients and/or adjuvants in the amounts indicated:
10 to 30 wt. %, preferably 15 to 25 wt. %, of binders, carriers and fillers, thereby replacing the water-soluble diluent;
0.1 to 5 wt. %, preferably 0.5 to 3 wt. %, of lubricants;
0.1 to 5 wt. %, preferably 0.3 to 2 wt. %, of flow control agents;
1 to 10 wt. %, preferably 2 to 8 wt. %, of crystallization retarders;
1 to 10 wt. %, preferably 2 to 8 wt. %, of solubilizers;
0.05 to 1.5 wt. %, preferably 0.1 to 0.8 wt. %, of coloring agents;
0.5 to 10 wt. %, preferably 2 to 8 wt. %, of pH control agents;
0.01 to 5 wt. %, preferably 0.05 to 1 wt. %, of surfactants and emulsifiers.

The second tablet layer composition comprises ramipril dispersed in a disintegrating tablet matrix having instant release (fast dissolution) characteristics. Alternatively, the second tablet layer composition comprises ramipril together with a diuretic dispersed in a disintegrating tablet matrix having instant release (fast dissolution) characteristics. The disintegrating tablet matrix may have weakly acidic, neutral or weakly basic properties, a neutral tablet matrix being preferred.

In a preferred embodiment, the disintegrating matrix comprises one or more fillers, a binder or polymer, a disintegrant, a lubricant and, optionally, other excipients and adjuvants.

Preferred fillers are selected from the group consisting of pregelatinized starch, microcristalline cellulose, low-substituted hydroxypropylcellulose, cellulose, mannitol, erythritol, lactose, saccharose, claciumhydrogenphosphate, sorbitol, and xylitol. Particularly preferred are pregelatinized starch, microcrystalline cellulose, mannitol and lactose monohydrate.

Preferred disintegrants are selected from the group consisting of croscarmellose sodium salt (cellulose carboxymethylether sodium salt, crosslinked), sodium starch glycolate, crosslinked polyvinyl pyrrolidone (crospovidone), corn starch and low-substituted hydroxypropylcellulose. Particularly preferred are sodium starch glycolate and croscarmellose sodium salt.

Preferred binders are selected from the group consisting of polyvinyl pyrrolidone (Povidone), copolymers of vinylpyrrolidone with other vinyderivatives (Copovidone), hydroxypropylmethylcellulose, methylcellulose, hydroxypropyl-cellulose and low-substituted hydroxypropyl-cellulose. Particularly preferred are hydroxypropyl-methylcellulose and Copovidone.

Preferred lubricants are sodium stearylfumarate and magnesium stearate.

The second tablet layer composition generally comprises 0.5 to 25 wt. %, preferably 1 to 15 wt. % of ramipril and 50 to 95wt. %, preferably 75 to 90 wt. % of fillers. The optional content of diuretic amounts to 2 to 15 wt. %.

The other excipients and/or adjuvants are, for instance, selected from binders, carriers, fillers, lubricants, flow control agents, crystallization retarders, solubilizers, coloring agents, pH control agents, surfactants and emulsifiers, specific examples of which are given below in connection with the third tablet layer composition. The excipients and/or adjuvants for the second tablet layer composition are preferably chosen such that a neutral, disintegrating tablet matrix is obtained. Examples for such fillers are mannitol, pregelatinized starch, lactose monohydrate and cellulose derivatives like low substituted hydroxypropylcellulose.

The optional third tablet layer composition contains a diuretic in a fast disintegrating tablet matrix. In a preferred embodiment, the disintegrating tablet matrix comprises a filler, a binder, a disintegrant and, optionally, other excipients and adjuvants.

The filler is preferably selected from anhydrous lactose, spray-dried lactose and lactose monohydrate.

The binder is selected from the group of dry binders and/or the group of wet granulation binders, depending on the manufacturing process chosen for the second tablet layer. Suitable dry binders are, e.g., cellulose powder and microcrystalline cellulose. Specific examples of wet granulation binders are corn starch, polyvinyl pyrrolidone (Povidone), vinylpyrrolidone-vinylacetate copolymer (Copovidone) and cellulose derivatives like hydroxymethylcellulose, hydroxyethylcellulose, hydroxypropyl-cellulose and hydroxypropylmethylcellulose.

Suitable disintegrants are, e.g., sodium starch glycolate, Crospovidon, Croscarmellose, sodium carboxymethylcellulose and dried corn starch, sodium starch glycolate being preferred.

The other excipients and adjuvants, if used, are preferably selected from diluents and carriers such as cellulose powder, microcrystalline cellulose, cellulose derivatives like hydroxymethylcellulose, hydroxyethylcellulose, hydroxypropylcellulose and hydroxy-propylmethylcellulose, dibasic calcium phosphate, corn starch, pregelatinized starch, polyvinyl pyrrolidone (Povidone) etc.; lubricants such as stearic acid, magnesium stearate, sodium stearylfumarate, glycerol tribehenate, etc.; flow control agents such as colloidal silica, talc, etc.; crystallization retarders such as Povidone, etc.; solubilizers such as Pluronic, Povidone, etc.; coloring agents, including dyes and pigments such as Iron Oxide Red or Yellow, titanium dioxide, talc, etc.; pH control agents such as citric acid, tartaric acid, fumaric acid, sodium citrate, dibasic calcium phosphate, dibasic sodium phosphate, etc.; surfactants and emulsifiers such as Pluronic, polyethylene glycols, sodium carboxymethyl cellulose, polyethoxylated and hydrogenated castor oil, etc.; and mixtures of two or more of these excipients and/or adjuvants.

In a particularly preferred embodiment the third layer is positioned between the first and second layer to avoid contact of telmisartan and ramipril with each other. The layers can be differentiated by using different colors.

Such a third tablet layer composition generally comprises 1.5 to 35 wt. %, preferably 2 to 15 wt. %, of active ingredient; 25 to 75 wt. %, preferably 35 to 65 wt. %, of filler; 10 to 40 wt. %, preferably 15 to 35 wt. %, of dry binder; 0.5 to 5 wt. %, preferably 1 to 4 wt. %, of wet granulation binder; and 1 to 10 wt. %, preferably 2 to 8 wt. %, of disintegrant. The other excipients and adjuvants are generally employed in the same amount as in the first tablet layer composition.

For preparing a bilayer tablet according to the present invention, the first and second tablet layer compositions may be compressed in the usual manner in a bilayer tablet press, e.g. a high-speed rotary press in a bilayer tableting mode. However, care should be taken not to employ an excessive compression force for the first tablet layer. Preferably, the ratio of the compression force applied during compression of the first tablet layer to the compression force applied during compression of both the first and second tablet layers is in the range of from 1:10 to 1:2. For instance, the first tablet layer may be compressed at moderate force of 4 to 8 kN, whereas the main compression of first plus second layer is performed at a force of 10 to 20 kN.

During bilayer tablet compression adequate bond formation between the two layers is achieved by virtue of distance attraction forces (intermolecular forces) and mechanical interlocking between the particles.

The multilayer tablets obtained release the active ingredients rapidly and in a largely pH-independent fashion, with complete release occurring within less than 60 min and release of the major fraction occurring within less than 15 min. The dissolution/-disintegration kinetics of the multilayer tablet may be controlled in different ways. For instance, the layers may dissolve/disintegrate simultaneously. Preferably, however, the second layer containing ramipril and the third tablet layer containing a diuretic disintegrate first whereas the first layer containing telmisartan dissolves subsequently.

In accordance with the present invention, a substantially increased dissolution rate of the active ingredients and, in particular, of telmisartan is achieved. Normally, at least 70% and typically at least 90% of the drug load are dissolved after 30 min.

The multilayer tablets of the present invention tend to be slightly hygroscopic and are therefore preferably packaged using a moisture-proof packaging material such as aluminium foil blister packs, or polypropylene tubes and high density polyethylene (HDPE) bottles which preferably contain a desiccant.

A preferred method of producing the bilayer tablet according to the present invention comprises:
(i) providing a first tablet layer composition by:
   a) preparing an aqueous solution of telmisartan, at least one basic agent and, optionally, a solubilizer and/or a crystallization retarder;
   b) spray-drying said aqueous solution to obtain a spray-dried granulate;
   c) mixing said spray-dried granulate with a water-soluble diluent to obtain a premix;
   d) mixing said premix with a lubricant to obtain a final blend for the first layer; and
   e) optionally, adding other excipients and/or adjuvants in any of steps a) to d);
(ii) providing a second tablet layer composition comprising ramipril alone or together with a diuretic;
(iii) optionally providing a third tablet layer composition comprising a diuretic;
(iv) compressing each of the first, second and third tablet layer composition to form a tablet layer; and
(v) compressing the separate tablet layers to form a multilayer tablet.

To provide a first tablet layer composition, an aqueous alkaline solution of telmisartan is prepared by dissolving the active ingredient in purified water with the help of one or more basic agents like sodium hydroxide and meglumine.

Optionally, a solubilizer and/or a recrystallization retarder may be added. The dry matter content of the starting aqueous solution is generally 10 to 40 wt. %, preferably 20 to 30 wt. %.

The aqueous solution is then spray-dried at room temperature or preferably at increased temperatures of, for instance, between 50° C. and 100° C. in a co-current or countercurrent spray-drier at a spray pressure of, for instance, 1 to 4 bar. Generally speaking, the spray-drying conditions are preferably chosen in such a manner that a spray-dried granulate having a residual humidity of ≦5 wt. %, preferably ≦3.5 wt. %, is obtained in the separation cyclone. To that end, the outlet air temperature of the spray-drier is preferably kept at a value of between about 80° C. and 90° C. while the other process parameters such as spray pressure, spraying rate, inlet air temperature, etc. are adjusted accordingly.

The spray-dried granulate obtained is preferably a fine powder having the following particle size distribution:
d₁₀: ≦20 μm, preferably ≦10 μm
d₅₀: ≦80 μm, preferably 20 to 55 μm
d₉₀: ≦350 μm, preferably 50 to 150 μm

After spray-drying, the active ingredient telmisartan as well as the excipients contained in the spray-dried granulate are in a substantially amorphous state with no crystallinity being detectable. From a physical point of view, the spray-dried granulate is a solidified solution or glass having a glass transition temperature Tg of preferably >50° C., more preferably >80° C.

Based on 100 parts by weight of active ingredient telmisartan, the spray-dried granulate preferably contains 5 to 200 parts by weight of basic agent and, optionally, solubilizer and/or crystallization retarder.

The water-soluble diluent is generally employed in an amount of 30 to 95 wt. %, preferably 60 to 80 wt. %, based on the weight of the first tablet layer composition.

The lubricant is generally added to the premix in an amount of 0.1 to 5 wt. %, preferably 0.3 to 2 wt. %, based on the weight of the first tablet layer composition. Mixing is carried out in two stages, i.e. in a first mixing step the spray-dried granulate and the diluent are admixed using, e.g., a high-shear mixer or a free-fall blender, and in a second mixing step the lubricant is blended with the premix, preferably also under conditions of high shear. The method of the invention is however not limited to these mixing procedures and, generally, alternative mixing procedures may be employed in steps c), d), and also in the subsequent steps f) and g), such as, e.g., container mixing with intermediate screening.

To provide a second tablet layer composition comprising ramipril alone, ramipril is premixed and granulated with a binder solution using a fluid bed granulator. Part of the excipients can be premixed and granulated together with ramipril in the fluid bed granulator. Optionally ramipril can be dissolved or suspended in the binder solution in order to improve content uniformity of ramipril in the final product. The dried granules are sieved through an appropriate sieve. After addition of the other excipients the mixture is blended in a free fall blender. Alternative methods for granulation of ramipril and excipients with the binder solution are high shear granulation or one pot granulation, followed by wet sieving, drying and dry sieving of the granules.

First and second tablet layer compositions as described above can be compressed into bilayer tablets of the target tablet weight with appropriate size and crushing strength, using an appropriate tablet press. Optionally, an appropriate external lubricant spray system for the dies and punches can be used during manufacturing of tablets in order to improve lubrication.

To provide an alternative second tablet layer composition comprising ramipril together with a diuretic such as hydrochlorothiazide (HCTZ), ramipril and hydrochlorothiazide are premixed and granulated together with part of the excipients with a binder solution in a fluid bed granulator as described above. Optionally, the active ingredients can be dissolved or suspended in the binder solution in order to improve content uniformity in the final product. After addition of the other excipients the mixture is blended in a free fall blender.

First and alternative second layer compositions as described above can be compressed into bilayer tablets with appropriate size and crushing strength, using an appropriate tablet press. Optionally, an appropriate external lubricant spray system for the dies and punches can be used during manufacturing of tablets in order to improve lubrication.

In a further embodiment, a third tablet layer composition comprising a diuretic may be prepared by dry-mixing the constituent components, e.g. by means of a high-intensity mixer or a free-fall blender. Alternatively and preferably, the third tablet layer composition is prepared using a wet granulation technique wherein an aqueous solution of a wet granulation binder is added to a premix and subsequently the wet granulate obtained is dried, e.g. in a fluidized-bed dryer or drying chamber. The dried mixture is screened and then a lubricant is admixed, e.g. using a tumbling mixer or free-fall blender.

First, second and third layer compositions as described above can be compressed into 3-layer tablets with appropriate size and crushing strength using an appropriate tablet press.

For the production of bilayer tablets according to the present invention, the separate tablet layer compositions can be compressed in a bilayer tablet press, e.g. a rotary press in the bilayer tableting mode, in the manner described above. In order to avoid any cross-contamination between the tablet layers (which could lead to decomposition of ramipril or HCTZ), any granulate residues have to be carefully removed during tableting by intense suction of the die table within the tableting chamber.

In order to further illustrate the present invention, the following non-limiting examples are given.

### FORMULATION EXAMPLES

### Example 1

### Telmisartan 80 mg/Ramipril 10 mg 2-Layer Tablets

| | | % of | % of |
|---|---|---|---|
| | | Telmisartan | Ramipril |
| Constituents | mg/tablet | layer | layer |
| Telmisartan | 80.000 | 16.667 | |
| Sodium hydroxide | 6.720 | 1.400 | |
| Povidone | 24.000 | 5.000 | |
| Meglumine | 24.000 | 5.000 | |
| Purified water * | 400.000 | | |
| Sorbitol | 337.280 | 70.267 | |
| Magnesium stearate | 8.000 | 1.667 | |
| Total Telmisartan layer | 480.000 | 100.000 | |
| Ramipril | 10.000 | | 5.000 |
| Microcrystalline cellulose | 60.000 | | 30.000 |
| Lactose monohydrate | 110.000 | | 55.000 |
| Hydroxypropyl methylcellulose | 6.000 | | 3.000 |
| Purified water * | 70.000 | | |
| Sodium starch glycolate | 8.000 | | 4.000 |
| Sodium stearyl fumarate | 6.000 | | 3.000 |
| Total Ramipril layer | 200.000 | | 100.000 |
| Total 2 layer tablet | 680.000 | | |

* Volatile component, does not remain in final product

### Example 2

### Telmisartan 80 mg/Ramipril 10 mg 2-Layer Tablets

| | | % of | % of |
|---|---|---|---|
| | | Telmisartan | Ramipril |
| Constituents | mg/tablet | layer | layer |
| Telmisartan | 80.000 | 16.667 | |
| Sodium hydroxide | 6.720 | 1.400 | |
| Povidone | 24.000 | 5.000 | |
| Meglumine | 24.000 | 5.000 | |
| Purified water * | 400.000 | | |
| Sorbitol | 337.280 | 70.267 | |
| Magnesium stearate | 8.000 | 1.667 | |
| Total Telmisartan layer | 480.000 | 100.000 | |
| Ramipril | 10.000 | | 5.000 |
| Microcrystalline cellulose | 80.000 | | 40.000 |
| Mannitol | 85.670 | | 42.835 |
| Hydroxypropyl methylcellulose | 10.000 | | 5.000 |
| Purified water * | 70.000 | | |
| Sodium starch glycolate | 8.000 | | 4.000 |
| Red iron oxide | 0.330 | | 0.165 |
| Sodium stearyl fumarate | 6.000 | | 3.000 |
| Total Ramipril layer | 200.000 | | 100.000 |
| Total 2 layer tablet | 680.000 | | |

* Volatile component, does not remain in final product

### Example 3

### Telmisartan 80 mg/Ramipril 5 mg 2-Layer Tablets

| | | % of | % of |
|---|---|---|---|
| | | Telmisartan | Ramipril |
| Constituents | mg/tablet | layer | layer |
| Telmisartan | 80.000 | 16.667 | |
| Sodium hydroxide | 6.720 | 1.400 | |
| Povidone | 24.000 | 5.000 | |
| Meglumine | 24.000 | 5.000 | |
| Purified water * | 400.000 | | |
| Sorbitol | 337.280 | 70.267 | |
| Magnesium stearate | 8.000 | 1.667 | |
| Total Telmisartan layer | 480.000 | 100.000 | |
| Ramipril | 5.000 | | 2.500 |
| Microcrystalline cellulose | 60.000 | | 30.000 |
| Lactose | 115.000 | | 57.500 |
| Hydroxypropyl methylcellulose | 6.000 | | 3.000 |
| Purified water * | 70.000 | | |
| Sodium starch glycolate | 8.000 | | 4.000 |
| Sodium stearyl fumarate | 6.000 | | 3.000 |
| Total Ramipril layer | 200.000 | | 100.000 |
| Total 2 layer tablet | 680.000 | | |

* Volatile component, does not remain in final product

### Example 4

### Telmisartan 80 mg/Ramipril 2.5 mg 2-Layer Tablets

| | | % of | % of |
|---|---|---|---|
| | | Telmisartan | Ramipril |
| Constituents | mg/tablet | layer | layer |
| Telmisartan | 80.000 | 16.667 | |
| Sodium hydroxide | 6.720 | 1.400 | |
| Povidone | 24.000 | 5.000 | |
| Meglumine | 24.000 | 5.000 | |
| Purified water * | 400.000 | | |
| Sorbitol | 337.280 | 70.267 | |
| Magnesium stearate | 8.000 | 1.667 | |
| Total Telmisartan layer | 480.000 | 100.000 | |
| Ramipril | 2.500 | | 1.250 |
| Microcrystalline cellulose | 60.000 | | 30.000 |
| Lactose | 117.500 | | 58.750 |
| Hydroxypropyl methylcellulose | 6.000 | | 3.000 |
| Purified water * | 70.000 | | |
| Sodium starch glycolate | 8.000 | | 4.000 |
| Sodium stearyl fumarate | 6.000 | | 3.000 |
| Total Ramipril layer | 200.000 | | 100.000 |
| Total 2 layer tablet | 680.000 | | |

* Volatile component, does not remain in final product

### Example 5

### Telmisartan 40 mg/Ramipril 5 mg 2-Layer Tablets

| | | % of | % of |
|---|---|---|---|
| | | Telmisartan | Ramipril |
| Constituents | mg/tablet | layer | layer |
| Telmisartan | 40.000 | 16.667 | |
| Sodium hydroxide | 3.360 | 1.400 | |
| Povidone | 12.000 | 5.000 | |
| Meglumine | 12.000 | 5.000 | |
| Purified water * | 200.000 | | |
| Sorbitol | 168.640 | 70.267 | |
| Magnesium stearate | 4.000 | 1.667 | |
| Total Telmisartan layer | 240.000 | 100.000 | |
| Ramipril | 5.000 | | 2.500 |
| Microcrystalline cellulose | 60.000 | | 30.000 |
| Lactose | 115.000 | | 57.500 |
| Hydroxypropyl methylcellulose | 6.000 | | 3.000 |
| Purified water * | 70.000 | | |
| Sodium starch glycolate | 8.000 | | 4.000 |
| Sodium stearyl fumarate | 6.000 | | 3.000 |
| Total Ramipril layer | 200.000 | | 100.000 |
| Total 2 layer tablet | 440.000 | | |

* Volatile component, does not remain in final product

### Example 6

### Telmisartan 80 mg/Ramipril 10 mg/HCTZ 12.5 mg 2-Layer Tablets

| | | % of | % of |
|---|---|---|---|
| | | Telmisartan | Ramipril + |
| Constituents | mg/tablet | layer | HCTZ layer |
| Telmisartan | 80.000 | 16.667 | |
| Sodium hydroxide | 6.720 | 1.400 | |
| Povidone | 24.000 | 5.000 | |
| Meglumine | 24.000 | 5.000 | |
| Purified water * | 400.000 | | |
| Sorbitol | 337.280 | 70.267 | |
| Magnesium stearate | 8.000 | 1.667 | |
| Total Telmisartan layer | 480.000 | 100.000 | |
| Ramipril | 10.000 | | 5.000 |
| Hydrochlorothiazide (HCTZ) | 12.500 | | 6.250 |
| Microcrystalline cellulose | 64.000 | | 32.000 |
| Mannitol | 93.170 | | 46.585 |
| Hydroxypropyl methylcellulose | 6.000 | | 3.000 |
| Purified water * | 70.000 | | |
| Sodium starch glycolate | 8.000 | | 4.000 |
| Red iron oxide | 0.330 | | 0.165 |
| Sodium stearyl fumarate | 6.000 | | 3.000 |
| Total Ramipril + HCTZ layer | 200.000 | | 100.000 |
| Total 2 layer tablet | 680.000 | | |

* Volatile component, does not remain in final product

### Example 7

### Telmisartan 80 mg/Ramipril 10 mg/HCTZ 12.5 mg 3-Layer Tablets

| | | % of | | |
|---|---|---|---|---|
| | | Telmisartan | % of HCTZ | % of Ramipril |
| Constituents | mg/tablet | layer | layer | layer |
| Telmisartan | 80.000 | 16.667 | | |
| Sodium hydroxide | 6.720 | 1.400 | | |
| Povidone | 24.000 | 5.000 | | |
| Meglumine | 24.000 | 5.000 | | |
| Purified water * | 400.000 | | | |
| Sorbitol | 337.280 | 70.267 | | |
| Magnesium stearate | 8.000 | 1.667 | | |
| Total Telmisartan layer | 480.000 | 100.000 | | |
| Hydrochlorothiazide (HCTZ) | 12.500 | | 8.333 | |
| Microcrystalline cellulose | 64.000 | | 42.667 | |
| Lactose monohydrate | 59.670 | | 39.780 | |
| Corn starch | 6.000 | | 4.000 | |
| Purified water * | q.s. | | | |
| Sodium starch glycolate | 6.000 | | 4.000 | |
| Red iron oxide | 0.330 | | 0.220 | |
| Magnesium stearate | 1.500 | | 1.000 | |
| Total HCTZ layer | 150.000 | | 100.000 | |
| Ramipril | 10.000 | | | 6.667 |
| Microcrystalline cellulose | 64.000 | | | 42.667 |
| Mannitol | 59.170 | | | 39.447 |
| Hydroxypropyl | 6.000 | | | 4.000 |
| methylcellulose | | | | |
| Purified water * | q.s. | | | |
| Yellow iron oxide | 0.330 | | | 0.220 |
| Sodium starch glycolate | 6.000 | | | 4.000 |
| Sodium stearyl fumarate | 4.500 | | | 3.000 |
| Total Ramipril layer | 150.000 | | | 100.000 |
| Total 3 layer tablet | 780.000 | | | |

* Volatile component, does not remain in final product

1. A pharmaceutical tablet comprising a first layer of telmisartan in a dissolving tablet matrix and a second layer of ramipril in a disintegrating tablet matrix.

2. The tablet of claim 1 additionally comprising a diuretic in the second layer having a disintegrating tablet matrix.

3. The tablet of claim 1 additionally comprising a diuretic in a separate third layer having a disintegrating tablet matrix.

4. The tablet of claim 3, wherein the third layer is positioned between the first and second layer and the layers are differentiated by using different colors.

5. The tablet of claim 1, wherein telmisartan is in a substantially amorphous form.

6. The tablet of claim 1, wherein the dissolving tablet matrix has instant release characteristics.

7. The tablet of claim 1, wherein the dissolving tablet matrix comprises a basic agent, a water-soluble diluent and, optionally, other excipients and adjuvants.

8. The tablet of claim 7, wherein the basic agent is selected from alkali metal hydroxides, basic amino acids and meglumine.

9. The tablet of claim 7, wherein the water-soluble diluent is selected from monosaccharides, oligosaccharides, and sugar alcohols.

10. The tablet of claim 9, wherein the water-soluble diluent is selected from glucose, sucrose, lactose, sorbitol, mannitol, and xylitol.

11. The tablet of claim 7, wherein the other excipients and adjuvants are selected from binders, carriers, fillers, lubricants, flow control agents, crystallization retarders, solubilizers, coloring agents, pH control agents, surfactants and emulsifiers.

12. The tablet of claim 1, wherein the first layer of telmisartan is produced by spray-drying an aqueous solution comprising telmisartan and a basic agent to obtain a spray-dried granulate; mixing said spray-dried granulate with a water-soluble diluent to obtain a premix; mixing said premix with a lubricant to obtain a final blend; and compressing the final blend to form the first tablet layer.

13. The tablet of claim 2, wherein the disintegrating tablet matrix of the second layer comprises a filler, a binder, a disintegrant and, optionally, other excipients and adjuvants.

14. The tablet of claim 3, wherein the disintegrating tablet matrix of the second or third layer comprises a filler, a binder, a disintegrant and, optionally, other excipients and adjuvants.

15. The tablet of claim 13, wherein the other excipients and adjuvants are selected from carriers, diluents, lubricants, flow control agents, solubilizers, coloring agents, pH control agents, surfactants and emulsifiers.

16. The tablet of claim 14, wherein the other excipients and adjuvants are selected from carriers, diluents, lubricants, flow control agents, solubilizers, coloring agents, pH control agents, surfactants and emulsifiers.

17. The tablet of claim 1, wherein the first layer contains 10-160 mg of telmisartan.

18. The tablet of claim 17, wherein the first layer contains 20-80 mg of telmisartan.

19. The tablet of claim 18, wherein the first layer contains 40-80 mg of telmisartan.

20. The tablet of claim 1, wherein the second layer contains 1-20 mg of ramipril and, optionally, 6.25 to 50 mg of hydrochlorothiazide.

21. The tablet of claim 20, wherein the second layer contains 5-10 mg of ramipril and, optionally, 12.5 to 25 mg of hydrochlorothiazide.

22. The tablet of claim 3, wherein the third layer contains 6.25 to 50 mg of hydrochlorothiazide.

23. The tablet of claim 22, wherein the third layer contains 12.5 to 25 mg of hydrochlorothiazide.

24. The tablet of claim 1 packaged in a moisture proof packaging material such as aluminium foil blister packs, or polypropylene tubes and high density polyethylene bottles.

25. The tablet of claim 24, wherein the moisture proof packaging material is aluminium foil blister packs, or polypropylene tubes and high density polyethylene bottles.

26. A method to treat or prevent a condition comprising administering the tablet of claim 1, wherein the condition is selected from the group consisting of stroke, myocardial infarction, transient ischaemic attack, cardiovascular disease, diabetes, cognitive decline and dementia.

27. A method to treat or prevent a condition comprising administering the tablet of claim 2, wherein the condition is selected from the group consisting of stroke, myocardial infarction, transient ischaemic attack, cardiovascular disease, diabetes, cognitive decline and dementia.

28. A method to treat or prevent a condition comprising administering the tablet of claim 3, wherein the condition is selected from the group consisting of stroke, myocardial infarction, transient ischaemic attack, cardiovascular disease, diabetes, cognitive decline and dementia.
